(19) United States
(12) Patent Application Publication
(10) Pub. No.: US 2026/0259121 A1
(43) Pub. Date: Sep. 3, 2026

(54) SYSTEMS, METHODS, AND DEVICES FOR NON-INVASIVE QUANTITATIVE ASSESSMENT OF MUCUS

(71) Applicants: Washington University, St. Louis, MO (US); Tufts Medical Center, Inc., Boston, MA (US)

(72) Inventors: Christine O'Brien, St. Louis, MO (US); Elizabeth Buzbee, St. Louis, MO (US); Michael House, Boston, MA (US); Alexandra Rutz, St. Louis, MO (US); Ashley Veade, St. Louis, MO (US); Jeannie Kelly, St. Louis, MO (US)

(21) Appl. No.: 19/552,698

(22) Filed: Feb. 27, 2026

Related U.S. Application Data
(60) Provisional application No. 63/764,987, filed on Feb. 28, 2025.

(51) Int. Cl. G01N 11/02; A61B 10/00; G01N 11/00; G01N 33/487

(52) U.S. Cl. CPC G01N 11/02; G01N 33/487; A61B 10/0096; G01N 2011/0006; G01N 2011/008

(57) ABSTRACT

A system for quantitative assessment of cervical mucus includes a laser speckle rheometer (LSR) and a computing device coupled to the LSR. The computing device includes a processor and a memory. The memory stores instructions that, when executed by the processor, configure the computing device to measure one or more rheologic property of a sample of a subject's cervical mucus using the LSR.

### CROSS REFERENCE TO RELATED APPLICATIONS

This application claims the benefit of priority to U.S. Provisional Patent Application No. 63/764,987, filed Feb. 28, 2025, which is hereby incorporated by reference in its entirety.

### STATEMENT REGARDING FEDERALLY SPONSORED RESEARCH & DEVELOPMENT

This invention was made with government support under TR002345 awarded by the National Institutes of Health. The government has certain rights in the invention.

### BACKGROUND

The field relates generally to the quantitative assessment of mucus, and more particularly to systems, methods, and devices for the non-invasive quantitative assessment of mucus.

Mucus is comprised of mucin glycoprotein polymers with many oligosaccharide chains that bind large amounts of water, enabling high water content (90-99% water) and unique viscoelastic properties. The mucins form disulfide bridges and hydrophobic cross-linking which restricts the amount of bound water and increases the stiffness of the mucus network. Highly viscous mucus is generally less permeable to micron-scale particles due to steric interactions with the highly concentrated and cross-linked mucus network.

Mucus properties are correlated with disease across many organ systems, including the reproductive tract, lungs, and gastrointestinal (GI) tract. In particular, mucus bulk viscoelasticity is known to be altered in many diseases including infertility, cystic fibrosis, and Crohn's Colitis. In respiratory diseases, highly viscous mucus inhibits normal mucociliary clearance and results in increased infection risk. A known cause of preterm birth (PTB) is intrauterine infection, caused by pathogens that ascend through the cervical canal to the sterile uterus. The cervical canal is filled with cervical mucus (CM) that serves as an immunological barrier to prevent passage of such pathogens. Patients at high risk of PTB have cervical mucus that is elastic, which corresponded with poor immunologic protection against ascending microbes, leaving patients susceptible to infection. CM may also play a crucial role in fertility. Its physical properties change significantly throughout the menstrual cycle due to hormonal fluctuations, influencing permeability and its barrier properties. In some cases of infertility, highly viscous CM prevents sperm penetration to the uterus.

There is limited research on how CM's properties relate to its permeability, particularly in the context of infection-mediated PTB and infertility. Some known studies have investigated the relationship between the mechanical properties of CM, permeability, and PTB risk using conventional rheology techniques. These require large sample volumes and costly equipment, making research progression challenging and limiting clinical translation.

The standard test for measuring bulk mucus viscoelastic properties is shear rheometry. This method measures the displacement of a fluid caused by a known shear force and rate and the results are divided into a storage modulus and loss modulus, which can be combined to measure the complex shear modulus. Shear rheometry systems are expensive (e.g., hundreds of thousands of dollars), bulky, require careful training, and need large sample volumes which are often not attainable for clinical mucus samples, thereby presenting significant barriers for measuring patient mucus specimens. Unsurprisingly, the biophysical properties of mucus are not routinely measured clinically despite known impact on many disease states, preventing the addition of important clinical biomarkers and measures of disease severity. A lack of clinically-translational tools that can measure mucus viscoelasticity has also severely restricted investigation of mucus outside of dedicated biopolymer and materials science research labs, limiting clinical research and mechanistic understanding of how mucus becomes altered in different adverse conditions.

This background section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

### BRIEF SUMMARY

According to one aspect of the present disclosure, a system for quantitative assessment of cervical mucus includes a laser speckle rheometer (LSR) and a computing device coupled to the LSR. The computing device includes a processor and a memory. The memory stores instructions that, when executed by the processor, configure the computing device to measure one or more rheologic property of a sample of a subject's cervical mucus using the LSR.

Another aspect of this disclosure is a laser speckle rheometer (LSR). The LSR includes a coherent light source to generate a beam of coherent light, a sample holder configured for containing a sample, a beam splitter positioned along a first optical axis between the coherent light source and the sample holder to allow the beam from the coherent light source to pass to the sample and to direct backscattered light reflected from the sample along a second optical axis different than the first optical axis, and a camera positioned along the second optical axis to receive backscattered light reflected from the sample and directed by the beam splitter, wherein at least one of the coherent light source and the sample holder is disposed at a non-zero angle relative to the first optical axis.

According to yet another aspect of the disclosure a method for non-invasive quantitative assessment of cervical mucus by measuring one or more rheologic property includes capturing a time-series of speckle images of a sample of a subject's cervical mucus using a laser speckle rheometer (LSR), quantifying a decay in a speckle pattern in the time-series of speckle images, determining a displacement of particles seen in the time-series of speckle images from an initial position, and determining a complex modulus of the sample based at least in part on the determined displacement.

Various refinements exist of the features noted in relation to the above-mentioned aspects. Further features may also be incorporated in the above-mentioned aspects. These refinements and additional features may exist individually or in any combination. For instance, various features discussed below in relation to any of the illustrated embodiments may be incorporated into any of the above-described aspects, alone or in any combination.

### BRIEF DESCRIPTION OF THE DRAWINGS

The following figures illustrate various aspects of the disclosure.

FIG. 1 is a simplified block diagram of an example system for quantitative assessment of cervical mucus (CM).

FIG. 2 is a diagram of another example system for quantitative assessment of CM.

FIG. 3 is a diagram of an example method for determining a rheologic property of a sample according to the present disclosure

FIG. 4 is a calibration curve for use as part of the method shown in FIG. 3.

FIG. 5 is a graph of the complex moduli measured with the system shown in FIG. 2 and a conventional rheometer for differently hydrated samples of synthetic mucus.

Corresponding reference characters indicate corresponding parts throughout the drawings.

### DETAILED DESCRIPTION

Embodiments of the present disclosure are generally directed to systems, methods, and devices for studying, assessing, and quantifying viscoelasticity of a sample. In some embodiments, the sample is a mucus sample from a subject. In some embodiments, the sample is, more particularly, a cervical mucus (CM) sample from the subject. For conciseness of description, the systems, methods, and devices of the present disclosure will be described with respect to the quantitative assessment of mucus, and more particularly to the non-invasive quantitative assessment of CM. It should be understood that the devices, systems, and methods of this disclosure may be used with any other suitable samples, including without limitation other hydrogels.

The example embodiments quantify physical properties of mucus, including mechanical properties, that can yield information about the permeability of mucus. Various embodiments include new uses and new device hardware.

Some embodiments of the present disclosure focus on examination of CM. The investigation of cervical mucus may be useful in, for example, fertility and preterm birth/pelvic infection. These example embodiments identify when CM is permeable, including permeability to sperm which would signal an optimized fertility window, or to infectious agents which will be applicable for preterm birth or pelvic infection risk assessment. Other embodiments are focused on other mucus, other biofluids, and/or other uses.

The example embodiments may have a high potential to improve patient outcomes. Example embodiments may identify patients at high-risk for infection-mediated preterm birth and enable timely administration of life-saving treatments such as antenatal corticosteroids and/or antibiotics to delay or prevent infection-mediated preterm birth. Increasing gestational age by even a few days can have significant impact on neonatal survival and decreasing neonatal morbidity if corticosteroids can be administered 24-48 hours prior to delivery. Corticosteroids significantly expedite development of the fetal lungs and reduce risk of brain and gastrointestinal tract complications post-delivery. The example embodiments may facilitate identification of risk factors for preterm birth, increased neonatal survival, reduced neonatal and long-term disability, decreased morbidity, and could change the standard of care for prenatal assessment of preterm birth risk. These may in turn result in decreased hospital length of stay, reduced hospital costs, reduction long-term morbidities associated with preterm birth, increased patient satisfaction, and improved patient-centered outcomes.

Example embodiments of the present disclosure include a small, low-cost laser speckle rheology (LSR) system capable of measuring the viscoelastic properties of small CM volumes. Laser speckle rheology works through Brownian motion detection. Particles in a sample move randomly within the sample based on the stiffness of the sample material. When the sample is illuminated by a coherent light source and the backscattered light is imaged, fluctuations in intensity of the images indicate the extent of Brownian motion. Greater intensity fluctuation is seen with a less stiff sample, while less intensity fluctuation is seen with a more stiff sample due to less random movement of particles in the stiff sample.

Some embodiments include a light-based platform that can provide quantitative results about CM mechanical properties in a low-cost, translational form factor that can help identify patients at high risk of preterm birth (PTB). The example systems provide accurate measurements of permeability and viscoelasticity for small CM volumes, overcoming the limitations of at least some known methods. Results from synthetic mucus experiments demonstrate the system's ability to measure both properties, while human CM studies should reveal the relationship between permeability and viscoelasticity across the menstrual cycle.

The example embodiments provide a novel, low-cost system for simultaneously measuring the mechanical properties of small CM volumes, enabling new insights into the relationship between CM barrier function and PTB risk. Additionally, the system is clinically transferable, presenting as a potential screening method for PTB risk assessment.

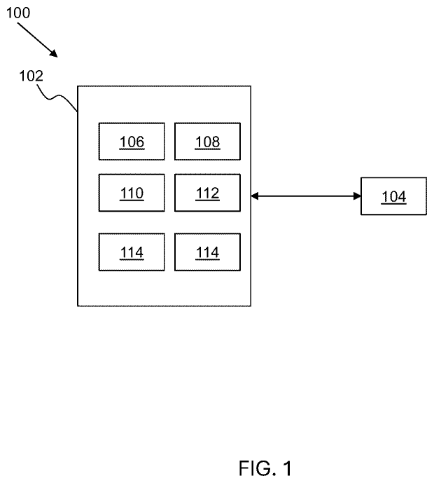

Turning now to the Figures, FIG. 1 is a simplified block diagram of an example system 100 for quantitative assessment of CM. The system includes a computing device 102 and a rheometer 104 communicatively coupled to the computing device. The communicative connection between the rheometer and the computing device may be any suitable wired or wireless connection. The computing device 102 may be or include any suitable stationary or portable computing device, computer, desktop computer, laptop computer, tablet computer, mobile device, single-board computer, microcontroller, microcomputer, system-on-module, programmable logic board, or the like. The computing device may also be referred to herein as “a controller”. In the example embodiment, the rheometer comprises a laser speckle rheometer.

The computing device 102 includes a processor 106, a memory 108, a media output component 110, an input device 112, and communications interfaces 114. Other embodiments include different components, additional components, and/or do not include all components shown in FIG. 1.

The processor 106 is configured for executing instructions. In some embodiments, executable instructions are stored in the memory 108. The processor 106 may include one or more processing units (e.g., in a multi-core configuration). As used herein, the term “processor” refers not only to integrated circuits, but also to a controller, a microcontroller, a microcomputer, a programmable logic controller (PLC), an application-specific integrated circuit, a graphic processing unit, and other programmable circuits. The memory 108 may generally be or include memory element(s) including, but not limited to, computer readable medium (e.g., random access memory (RAM)), computer readable non-volatile medium (e.g., a flash memory), a floppy disk, a compact disc-read only memory (CD-ROM), a magneto-optical disk (MOD), a digital versatile disc (DVD) and/or other suitable non-transitory memory elements and is generally any device allowing information such as executable instructions and/or other data to be stored and retrieved. Such memory 108 may generally be configured to store suitable computer-readable instructions that, when implemented by the processor 106, configure, cause, or program the computing device 102 to perform various functions described herein.

The media output component 110 is configured for presenting information to a user (not shown). The media output component 110 is any component capable of conveying information to the user. In some embodiments, the media output component 110 includes an output adapter such as a video adapter and/or an audio adapter. The output adapter is operatively connected to the processor 106 and operatively connectable to an output device such as a display device (e.g., a liquid crystal display (LCD), organic light emitting diode (OLED) display, cathode ray tube (CRT), “electronic ink” display, one or more light emitting diodes (LEDs)) or an audio output device (e.g., a speaker or headphones).

The computing device 102 includes, or is connected to, the input device 112 for receiving input from the user. The input device is any device that permits the computing device 102 to receive analog and/or digital commands, instructions, or other inputs from the user, including visual, audio, touch, button presses, stylus taps, etc. The input device 112 may include, for example, a variable resistor, an input dial, a keyboard/keypad, a pointing device, a mouse, a stylus, a touch sensitive panel (e.g., a touch pad or a touch screen), a gyroscope, an accelerometer, a position detector, or an audio input device. A single component such as a touch screen may function as both an output device of the media output component 110 and the input device 112.

The communication interfaces 114 enable the computing device 102 to communicate with remote devices and systems, such as allowing communication between the computing device 102 and the rheometer 104, remote computing devices or servers (not shown), and the like. The communication interfaces 114 may be wired or wireless communications interfaces that permit the computing device to communicate with the remote devices and systems directly or via a network. Wireless communication interfaces 114 may include a radio frequency (RF) transceiver, a Bluetooth® adapter, a Wi-Fi transceiver, a ZigBee® transceiver, a near field communication (NFC) transceiver, an infrared (IR) transceiver, and/or any other device and communication protocol for wireless communication. (Bluetooth is a registered trademark of Bluetooth Special Interest Group of Kirkland, Washington; ZigBee is a registered trademark of the ZigBee Alliance of San Ramon, California.) Wired communication interfaces 114 may use any suitable wired communication protocol for direct communication including, without limitation, USB, RS232, I2C, SPI, analog, and proprietary I/O protocols. In some embodiments, the wired communication interfaces 114 include a wired network adapter allowing the computing device to be coupled to a network, such as the Internet, a local area network (LAN), a wide area network (WAN), a mesh network, and/or any other network to communicate with remote devices and systems via the network. Although two communication devices 114 are shown, the computing device 102 may include more or fewer computing devices.

It should be understood that in some embodiments the computing device 102 does not include or use an input device 112 or a media output 110 and a user may not directly interact with the computing device. Rather, the user (or another computing device) may only interact remotely with computing device 102 through the communication interface 114.

Moreover, in some embodiments the computing device 102, or parts thereof, may not be a physical computing device local to the user, but instead is cloud based. Thus, for example, the computing device 102 may be a cloud-based computing device or may be a physical computing device 102 using cloud-based storage for all or part of its memory, using cloud-based processing instead of local processing for some or all of its processing, or the like. Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. As used herein, the term “cloud computing” and related terms, e.g., “cloud computing devices” refers generally to a computer architecture allowing for the use of multiple heterogeneous computing devices for data storage, retrieval, and processing. The heterogeneous computing devices may use a common network or a plurality of networks so that some computing devices are in networked communication with one another over a common network but not all computing devices. In other words, a plurality of networks may be used to facilitate the communication between and coordination of all computing devices.

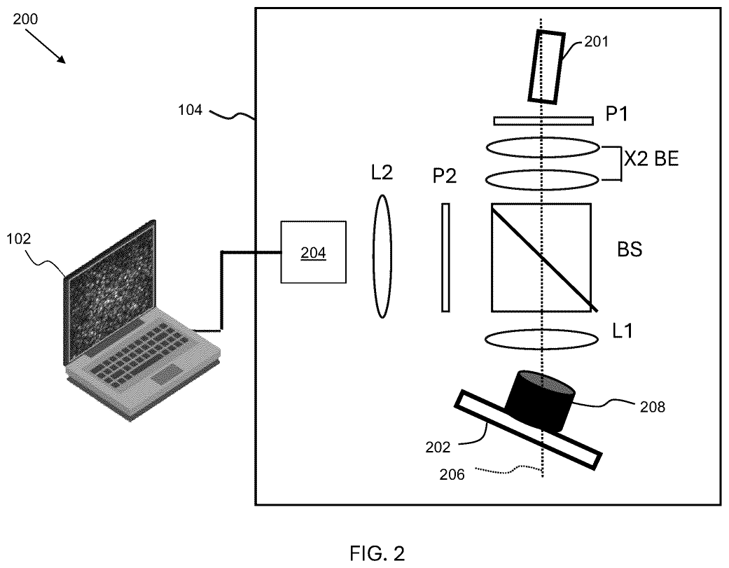

FIG. 2 is a diagram of another example system 200 for quantitative assessment of CM. The system 200 operates similar to the system 100 and components identified by the same reference numbers are similar components that operate similarly unless described otherwise. In the system 200, the computing device 102 is a laptop computing device and the rheometer 104 is a laser speckle rheometer.

The rheometer 104 includes a coherent light source 201, polarizers P1 and P2, a beam expander X2 BE, lenses L1 and L2, a translational stage 202, and a camera 204. In the example embodiment, the coherent light source 201 is a 780 nm laser with a built-in collimator. Other embodiments may use a laser emitting a beam at any other suitable wavelength or any other suitable coherent light source. The laser is positioned at a 2° angle relative to the optical axis 206 to facilitate minimizing specular reflection back into the camera 204. In some other embodiments, different coherent light sources are used, different wavelength lasers are used, and/or the laser is positioned at a different nonzero angle relative to the optical axis or at a 0° angle (e.g., to direct the beam along/parallel to the optical axis). In some embodiments, a variable iris diaphragm aperture (not shown) is incorporated directly below the laser output to optimize the speckle image quality and minimize unwanted reflections. The light output from the laser travels through the first polarizer P1 and into a Galilean beam expander X2 BE that provides 2× magnification. In the example embodiment, the beam expander X2 BE is implemented with lenses of −75 mm and 150 mm focal lengths. In still other embodiments, different magnification, including no magnification, may be used. A beam splitter BS directs the laser light to the sample arm, where it is focused using first lens L1 onto a sample. The first lens L1 has a focal length of 50 mm in the example embodiment.

The sample is placed in a sample well 208 mounted to a glass slide (not shown), which collectively may be referred to as a sample holder. In the example embodiment, the sample well is a 3D-printed well fabricated from matte black polylactic acid (PLA) to minimize stray light reflections. The example well is 2 mm deep with a standard volume capacity of 1 mL. Other embodiments use sample wells sized for larger or smaller sample volumes made from other materials, and/or made using different fabrication techniques. The sample in the well 208 is covered with a glass coverslip to ensure a consistent optical path length. The sample well 208 and the glass slide are mounted to the translational stage 202. The translational stage 202 is mounted at 20° from perpendicular to the optical axis 206, creating a net 18° angle (2° laser+20° stage) to further reduce specular reflection from the sample surface back into the detection path. In other embodiments, the translational stage 202 is different nonzero angle relative to the optical axis or at a 0° angle. The translational stage 202 is an XY translation stage to allow precise positioning of the sample relative to the light from the laser. In some embodiments, a motorized system is used to enable consistent scanning on samples, e.g., by moving the translational stage using motors controlled by the computing device. In addition to angling the laser and the sample well, the specular reflectance that impacts results of system may be further limited by one or more of the addition of apertures to control the level of incident light, the construction of the sample well, using a polarization sensitive camera, and using a cage system/rails. Further features of the processing pipeline discussed below may also reduce the impact of reflections.

In some embodiments, the samples are collected by syringe (speculum-based), a hydrophobic polyurethane foam brush (cervico-vaginal based), or a disc or cup that is inserted into the vaginal canal to collect cervical mucus that is discharged from the cervix. To prevent de-hydration of the samples due to hydrophilic interactions with the collection tools, the following sample preparation and loading protocol is used. A portion of each collected sample will be transferred into a microfuge tube that has a desired volume delineated thereon; gel will be eluted by expelling contents from the syringe or scraping contents from the foam brush on the side of the microfuge tube until the mucus reaches the volume line, after which no additional gel will be added. The sample will be extracted from the microfuge tube with a pipette and transferred to the sample well 208. A coverslip is placed above the gel with gentle pressure; excess gel will be carefully squeezed out of the well until the coverslip is in contact with the top edges of the well. The loaded sample well 208 mounted to a glass slide and covered by the coverslip is then mounted to the translational stage 202 for assessment by the system.

When the sample is illuminated by the light from the laser, backscattered light is reflected from the sample and directed via the beam splitter BS toward the camera 204. The reflected light first passes through the second polarizer P2 and the second lens L2 before reaching the camera 204. The second lens L2 has a focal length of 44 mm in the example embodiment. The second lens L2 focuses the backscattered light onto the sensor of the camera 204, optimizing the numerical aperture (NA) to ensure the speckle size (S) is at least twice the camera sensor's pixel size (4.8 μm×4.8 μm in the example). The speckle size follows Equation 1:

$$S = 2.44 λ ( 1 + M ) f / \# \qquad ( 1 )$$

where λ is the laser wavelength (780 nm in the example embodiment), M is the system magnification, and f/#=f/NA, where f is the focal length. To maintain an adequate speckle size, NA greater than 2.8 is preferred for the example embodiment. In other embodiments NA may be different, particularly when magnification differs from the example embodiment. The camera 204 images the speckle pattern reflected from the sample. In the example embodiment, the camera 204 is a high-speed monochrome camera capable of capturing more than 100 frames per second. In some embodiment, the camera 204 is operable to capture more than 200, more than 300, more than 400, or more than 500 frames per second. In some embodiments, the camera is operable to capture 550 frames per second.

In some embodiments, the camera 204 is a polarization sensitive camera and the first and second polarizers P1, P2 are not included in the system.

The images of the speckle pattern reflected from the sample are output from the camera 204 to the computing device 102. As will be described in further detail below, the computing device determines one or more rheologic properties of the sample from the received images. In some embodiments, the computing device determines a recommendation or an assessment based at least in part on the determined rheologic properties of the sample or the received images.

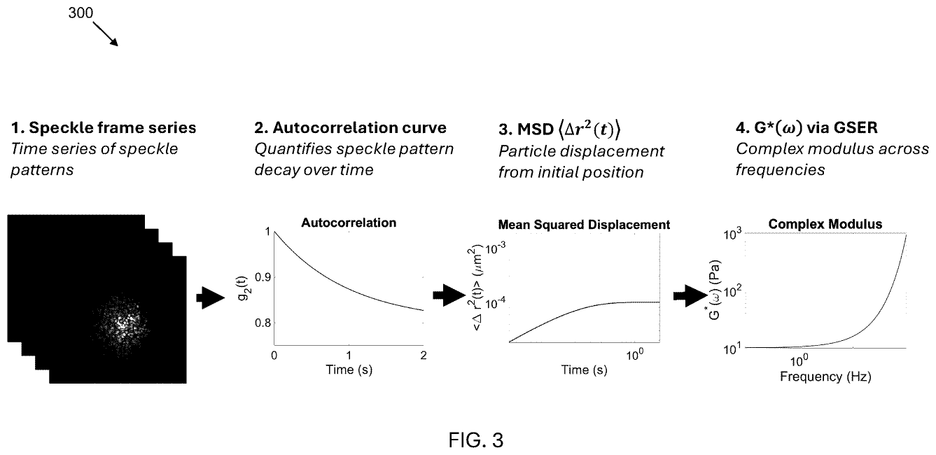

FIG. 3 is a diagram of an example method 300 determining a rheologic property of a sample according to the present disclosure. The method 300 may be performed by the computing device 102 in the system 100 or 200. The method will be described with respect to system 200 but may be performed by system 100 or any other suitable system.

The first step of the method 300 is capturing a time series of speckle images. The computing device 102 controls the laser speckle rheometer 104 to cause the camera 204 to capture a plurality of laser speckle images of a sample over a period of time. In some embodiments, the period of time is on the order of seconds of time. In an embodiment, the period of time is about three seconds. In other embodiments, the period of time longer or shorter, including less than one second. This time series of speckle images is output from the camera 204 to the computing device 102.

Next, the computing device 102 performs temporal autocorrelation to quantify how the pixels in the speckle images decorrelate over time by cross-correlating a first speckle image with temporally subsequent speckle images. In the example embodiment, an autocorrelation curve is determined by:

$${g}_{2} ( t ) = {〈 \frac{{〈 I ( {t}_{0} ) I ( {t}_{0} + t ) 〉}_{pixels}}{\sqrt{{〈 {I ( {t}_{0} )}^{2} 〉}_{pixels} {〈 {I ( {t}_{0} + t )}^{2} 〉}_{pixels}}} 〉}_{{t}_{0}} \qquad ( 2 )$$

where I(t₀) represents the initial intensity, and I(t₀+t) corresponds to the intensity in subsequent frames. This equation quantifies the decorrelation of pixel intensities over time. To accurately measure the bulk modulus, a spatially averaged pixel window is used in the example embodiment. The window size is adjusted according to the dimensions of the speckle image, ensuring it is large enough to encompass the scattered light pattern. In samples with low scattering, a smaller window may be employed to exclude dark pixels from the g₂(t) curve calculation, thereby preventing inaccuracies. Other embodiments may use any other suitable autocorrelation function. Faster decay of this curve implies higher random motion of the particles and a lower stiffness of the sample, while slower decay implies lower random motion of the particles and a higher stiffness of the sample.

Next, this temporal information is converted into spatial information by calculating the mean squared displacement (MSD) of particles from their initial positions. In physics, MSD is a measure of how much a particle has moved from its initial position over time. It is essentially the average of the squared distance a particle travels, providing insights into the nature of particle motion, especially in systems like diffusion or random walks. In method 300, the determined MSD is a measure of how much a particle has moved from its initial position over time and is influenced by the absorption coefficient, which is a measure of how strongly a material absorbs light and the reduced scattering coefficient which describes how light scatters in a sample.

The MSD of scattering particles can be calculated from the autocorrelation function g₂(t) previously determined. The appropriate equation for calculating MSD depends on the magnitude of absorption relative to scattering. Equations 3a and 3b, describe the MSD calculations under different absorption conditions:

$$If 0 < \frac{{μ}_{a}}{{μ}_{s}^{′}} < 0.1 : {g}_{2} ( t ) = {e}^{- 2 γ} \sqrt{{k}^{2} 〈 Δ {r}^{2} ( t ) 〉 + \frac{3 {μ}_{a}}{{μ}_{s}^{′}}} \qquad ( 3 a )$$

$$else if {μ}_{a} = 0 : {g}_{2} ( t ) = {e}^{- 2 {γ ( {k}^{2} 〈 Δ {r}^{2} ( t ) 〉 )}^{ζ}} \qquad ( 3 b )$$

where k is the wavenumber (k=2π/λ) and <Δr²(t)> represents the MSD. The optical parameters γ and ζ are experimentally-derived constants that depend on the optical properties (μₐ and μₛ′) of the sample and are used in the diffusion wave spectroscopy (DWS) formalism to relate g₂(t) to MSD. These parameters account for the complex light propagation behavior in turbid media and cannot be calculated analytically for arbitrary optical properties. Instead, they must be determined empirically for the specific optical configuration of the system.

In the example embodiment, the optical properties of samples (absorption coefficient μₐ and reduced scattering coefficient μₛ′) were determined experimentally by measuring the radial diffuse reflectance profile (DRP) from time-averaged speckle images and fitting to the Farrell diffusion model. This approach provides a direct measurement of the optical properties from the same speckle data used for rheological analysis, eliminating the need for separate integrating sphere measurements in the final pipeline. The radial DRP was obtained by radially averaging the intensity of the time-averaged speckle image as a function of distance from the illumination center. This profile was then fitted to the steady-state diffusion model to extract μₐ and μₛ′.

A lookup table (e.g., stored in memory 108) maps optical properties to their corresponding γ and ζ values. To generate the lookup table, Monte Carlo simulations were performed using MCX (Monte Carlo eXtreme) software. In one embodiment, a total of 200 combinations of optical properties were simulated, spanning the range expected for cervical mucus and synthetic mucus samples. Other embodiments may simulate any suitable number of combinations of optical properties. In an example embodiment, the absorption coefficient μₐ ranged from approximately 10⁻¹³ to 10⁻⁴ mm⁻¹, while the reduced scattering coefficient μₛ′ ranged from approximately 0.2 to 7.5 mm⁻¹. In another embodiment, μₐ ranged from approximately 0.0-0.5 mm⁻¹, while μₛ′ ranged from approximately 0.01-8.0 mm⁻¹. For each combination, photon propagation was simulated through a virtual sample matching the geometry of the system 200, and the resulting time-varying speckle patterns were generated.

Autocorrelation analysis was then performed on the simulated speckle sequences by tracking the momentum transfer to calculate g₂(t) curves for each set of optical properties. By fitting these curves to the DWS formalism (Equations 3a and 3b), the corresponding γ and ζ parameters were extracted. The resulting lookup table establishes the relationship between (μₐ, μₛ′) pairs and (γ, ζ) pairs specific to the system geometry. During analysis of experimental data, the measured optical properties are used to interpolate the appropriate γ and ζ values from this lookup table. This Monte Carlo-based approach ensures accurate accounting of the optical scattering effects specific to the system configuration, improving the precision of MSD calculations.

To solve for MSD, the previously determined g₂(t) function is used along with the corresponding optical properties and optical parameters. Equation 3a follows the DWS formalism for absorbing media, while Equation 3b is used when μₐ is negligible and represents a modified formalism based on Monte Carlo simulations. The MSD curve <Δr²(t)> as a function of time provides critical information about particle dynamics within the viscoelastic medium and serves as input to the generalized Stokes-Einstein relation for calculating the complex modulus.

Next, the computing device 102 determines the complex modulus of the sample. The complex modulus can then be determined by substituting the calculated MSD curve over increasing frequencies into the Generalized Stokes-Einstein Relation (GSER):

$${G}^{*} ( ω ) = \frac{{K}_{B} T}{π a Γ [ 1 + α ( t ) ] Δ {r}^{2} ( t )} {\vert}_{t = 1 / ω} \qquad ( 4 )$$

where a is the particle size determined from an azimuthal diffuse reflectance calibration curve, K_{B} is the Boltzmann constant (1.38×10⁻²³ J/K), T is the absolute temperature in Kelvin, ω is the angular frequency, and α is the log-log slope of the MSD curve. The gamma function is denoted by P. This equation relates the microscopic particle dynamics (captured in the MSD) to the macroscopic viscoelastic properties of the material (represented by G*). The complex shear modulus G* can be decomposed into its real component (storage modulus G′) and imaginary component (loss modulus G″), providing comprehensive information about the elastic and viscous properties of the sample across a range of frequencies.

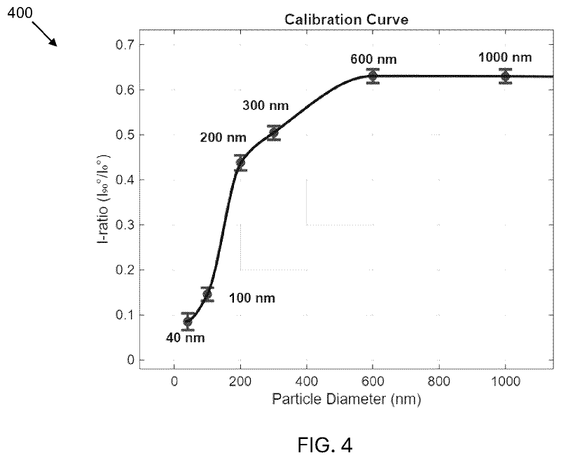

In the example embodiment, the mean optical scatterer size (a) is estimated experimentally. using the azimuthal diffuse reflectance profile method. In other embodiments, literature values or any other suitable estimate for mucin size may be used. The azimuthal diffuse reflectance profile is obtained by measuring the ratio of light intensity along two orthogonal axes of the diffuse reflectance pattern (I(φ=90°)/I(φ=0°)). This ratio, denoted as Î, has been shown to correlate strongly with scatterer size. To establish a calibration curve for the example embodiment, polystyrene beads of known sizes (40 nm, 100 nm, 200 nm, 300 nm, 600 nm, and 1 μm diameter) were suspended in water and imaged using the system 200. For each bead size, the azimuthal intensity ratio Î was calculated and plotted against the known bead diameter to produce a calibration curve 400 shown in FIG. 4. The calibration curve 400 follows the expected trends, with Î increasing as particle size increases. For experimental cervical mucus or synthetic mucus samples, the azimuthal intensity ratio is measured from the speckle images using a parallel polarized configuration (all other analysis is done using cross polarized images), and the scatterer size is estimated by comparing this ratio to the calibration curve. This experimentally-determined scatterer size is then used as the parameter a in the GSER calculation.

When determining the scatterer size, images were segmented into the four quadrants. The user chooses which quadrants are used to be averaged for the azimuthal DRP. This technique is used because different images have different reflections, and it allows for a more accurate readout when quadrants without reflections were chosen. Additionally, the calibration curve was developed using experimental data (beads of known sizes) rather than simulation.

The computing device 102 next generates an output based at least in part on the determined complex modulus for the sample being analyzed by the system 200. In some embodiments, the output is the complex modulus. In some embodiments, the real and imaginary components of the complex modulus are additionally or alternatively output. In some embodiments a further determination or recommendation is output in addition or alternatively to the complex modulus and/or its components. For example, the computing device 102 may compare the results to previous results from samples from the same subject from different times, to thresholds determined based on a population of subjects, or input the results to a trained deep learning algorithm to determine a further determination or recommendation. For example, when the system is being used for fertility assistance, the computing device 102 may determine whether the subject is more or less fertile currently, may determine a preferred time to attempt to conceive, or the like. The determination may be output as a numerical value, a categorization in words or symbols (e.g., “more likely to conceive”, “less likely to conceive”, a green/yellow/red indication, or the like). When the system 200 is used in the field of PTB, the computing device 102 may determine and output a risk of infection and/or likelihood of PTB. Again, the output may be a numerical output, a relative/categorical output, or the like.

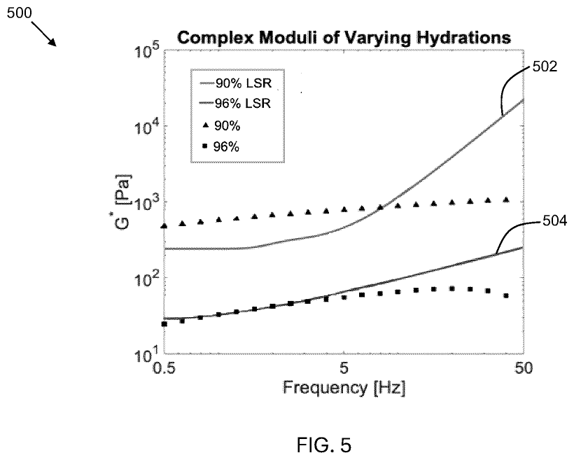

A prototype of the system 200 was constructed and tested. Specifically, samples of synthetic mucus at 90% hydration and 96% percent hydration were analyzed using the system 200 and a conventional rheometer. FIG. 5 is a graph 500 of the complex moduli measured with the system 200 and a conventional rheometer for the differently hydrated samples of synthetic mucus. Complex moduli measured with system 200 are shown as solid lines (trace 502 for 90% hydration and trace 504 for 96% hydration), while discrete points show results from a conventional rheometer. The intraclass correlation coefficient, which measures agreement between two datasets (0=none, 1=perfect), was above 0.9 for both hydrations (0.9152 for 90% hydration and 0.9414 for 96% hydration), indicating strong agreement with the gold standard. These trials were performed before incorporating scatterer size and optical property measurements as described above and the values were estimated from literature or alternative systems for the purposes of these tests. Integrating the measured values as described above will improve the system accuracy and better capture the frequency-dependent trends seen in the rheometer data.

This written description uses examples to disclose the invention, including the best mode, and also to enable any person skilled in the art to practice the invention, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

As used herein, the terms “about,” “substantially,” “essentially” and “approximately” when used in conjunction with ranges of dimensions, concentrations, temperatures or other physical or chemical properties or characteristics is meant to cover variations that may exist in the upper and/or lower limits of the ranges of the properties or characteristics, including, for example, variations resulting from rounding, measurement methodology or other statistical variation.

When introducing elements of the present disclosure or the embodiment(s) thereof, the articles “a”, “an”, “the” and “said” are intended to mean that there are one or more of the elements. The terms “comprising,” “including,” “containing” and “having” are intended to be inclusive and mean that there may be additional elements other than the listed elements. The use of terms indicating a particular orientation (e.g., “top”, “bottom”, “side”, etc.) is for convenience of description and does not require any particular orientation of the item described.

As various changes could be made in the above constructions and methods without departing from the scope of the disclosure, it is intended that all matter contained in the above description and shown in the accompanying drawing[s] shall be interpreted as illustrative and not in a limiting sense.

What is claimed is:

1. A system for quantitative assessment of cervical mucus, the system comprising:
a laser speckle rheometer (LSR); and
a computing device coupled to the LSR, the computing device including a processor and a memory, the memory storing instructions that, when executed by the processor, configure the computing device to measure one or more rheologic property of a sample of a subject's cervical mucus using the LSR.

2. The system of claim 1, wherein the instructions configure the computing device to measure one or more rheologic property of the sample of the subject's cervical mucus by:
capturing a time-series of speckle images of the sample using the LSR;
quantifying a decay in a speckle pattern in the time-series of speckle images;
determining a displacement of particles seen in the time-series of speckle images from an initial position; and
determining a complex modulus of the sample based at least in part on the determined displacement.

3. The system of claim 2, wherein the instructions configure the computing device to quantify the decay in the speckle pattern in the time-series of speckle images by:
determining an autocorrelation curve by cross-correlating a first speckle image of the time-series of speckle images with temporally subsequent speckle images of the time-series of speckle images to quantify how pixels in the speckle images decorrelate over time.

4. The system of claim 3, wherein the instructions configure the computing device to determine the autocorrelation curve as:
$${g}_{2} ( t ) = {〈 \frac{{〈 I ( {t}_{0} ) I ( {t}_{0} + t ) 〉}_{pixels}}{\sqrt{{〈 {I ( {t}_{0} )}^{2} 〉}_{pixels} {〈 {I ( {t}_{0} + t )}^{2} 〉}_{pixels}}} 〉}_{{t}_{0}}$$
where I(t₀) represents an initial intensity, and I(t₀+t) corresponds to an intensity in subsequent speckle images.

5. The system of claim 4, wherein the instructions configure the computing device to determine the displacement of particles seen in the time-series of speckle images from the initial position by calculating mean squared displacement (MSD) of particles from their initial positions.

6. The system of claim 5, wherein the instructions configure the computing device to calculate an MSD curve under different absorption conditions using
$$If 0 < \frac{{μ}_{a}}{{μ}_{s}^{′}} < 0.1 : {g}_{2} ( t ) = {e}^{- 2 γ} \sqrt{{k}^{2} 〈 Δ {r}^{2} ( t ) 〉 + \frac{3 {μ}_{a}}{{μ}_{s}^{′}}}$$
$$else if {μ}_{a} = 0 : {g}_{2} ( t ) = {e}^{- 2 {γ ( {k}^{2} 〈 Δ {r}^{2} ( t ) 〉 )}^{ζ}}$$
where k is a wavenumber equal to 2π/λ, <Δr²(t)> represents the MSD, γ and ζ are experimentally-derived constants derived absorption coefficient μₐ and reduced scattering coefficient μₛ′.

7. The system of claim 6, wherein the instructions configure the computing device to determine the complex modulus of the sample by substituting the calculated MSD curve over increasing frequencies into Generalized Stokes-Einstein Relation (GSER):
$${G}^{*} ( ω ) = \frac{{K}_{B} T}{π a Γ [ 1 + α ( t ) ] Δ {r}^{2} ( t )} {\vert}_{t = \frac{1}{ω}}$$
where a is a particle size determined from an azimuthal diffuse reflectance calibration curve, K_{B} is Boltzmann constant (1.38×10⁻²³ J/K), T is absolute temperature in Kelvin, ω is an angular frequency, and α is a log-log slope of the calculated MSD curve.

8. The system of claim 1, wherein the instructions further configure the computing device to output the measured one or more rheologic property.

9. The system of claim 1, wherein the instructions further configure the computing device to:
calculate a determination or recommendation based at least in part on the measured one or more rheologic property; and
output the determination or recommendation.

10. A laser speckle rheometer (LSR) comprising:
a coherent light source to generate a beam of coherent light;
a sample holder configured for containing a sample;
a beam splitter positioned along a first optical axis between the coherent light source and the sample holder to allow the beam from the coherent light source to pass to the sample and to direct backscattered light reflected from the sample along a second optical axis different than the first optical axis; and
a camera positioned along the second optical axis to receive backscattered light reflected from the sample and directed by the beam splitter, wherein at least one of the coherent light source and the sample holder is disposed at a non-zero angle relative to the first optical axis.

11. The LSR of claim 10, wherein the coherent light source is disposed at a two degree angle relative to the first optical axis and the sample holder is disposed at a twenty degree angle relative to the first optical axis.

12. The LSR of claim 10, further comprising a translational stage supporting the sample holder.

13. The LSR of claim 10, wherein coherent light source comprises a laser.

14. The LSR of claim 10, further comprising:
at least one first lens positioned along the first optical axis between the coherent light source and the sample holder to focus the beam onto the sample; and
at least one second lens positioned along the second optical axis between the beam splitter and the camera to focus backscattered light reflected from the sample onto a sensor of the camera.

15. The LSR of claim 10 further comprising at least one polarizing filter positioned along one or both of the first optical axis and the second optical axis.

16. A method for non-invasive quantitative assessment of cervical mucus by measuring one or more rheologic property, the method comprising:
capturing a time-series of speckle images of a sample of a subject's cervical mucus using a laser speckle rheometer (LSR);
quantifying a decay in a speckle pattern in the time-series of speckle images;
determining a displacement of particles seen in the time-series of speckle images from an initial position; and
determining a complex modulus of the sample based at least in part on the determined displacement.

17. The method of claim 16, wherein quantifying the decay in the speckle pattern in the time-series of speckle images comprise determining an autocorrelation curve as:
$${g}_{2} ( t ) = {〈 \frac{{〈 I ( {t}_{0} ) I ( {t}_{0} + t ) 〉}_{pixels}}{\sqrt{{〈 {I ( {t}_{0} )}^{2} 〉}_{pixels} {〈 {I ( {t}_{0} + t )}^{2} 〉}_{pixels}}} 〉}_{{t}_{0}}$$
where I(t₀) represents an initial intensity, and I(t₀+t) corresponds to an intensity in subsequent speckle images.

18. The method of claim 17, wherein determining the displacement of particles seen in the time-series of speckle images from the initial position comprises calculating an MSD curve under different absorption conditions using
$$If 0 < \frac{{μ}_{a}}{{μ}_{s}^{′}} < 0.1 : {g}_{2} ( t ) = {e}^{- 2 γ} \sqrt{{k}^{2} 〈 Δ {r}^{2} ( t ) 〉 + \frac{3 {μ}_{a}}{{μ}_{s}^{′}}}$$
$$else if {μ}_{a} = 0 : {g}_{2} ( t ) = {e}^{- 2 {γ ( {k}^{2} 〈 Δ {r}^{2} ( t ) 〉 )}^{ζ}}$$
where k is a wavenumber equal to 2π/λ, <Δr² (t)> represents the MSD, γ and ζ are experimentally-derived constants derived absorption coefficient μₐ and reduced scattering coefficient μₛ′.

19. The method of claim 18, wherein determining the complex modulus of the sample comprises substituting the calculated MSD curve over increasing frequencies into Generalized Stokes-Einstein Relation (GSER):
$${G}^{*} ( ω ) = \frac{{K}_{B} T}{π a Γ [ 1 + α ( t ) ] Δ {r}^{2} ( t )} {\vert}_{t = \frac{1}{ω}}$$
where a is a particle size determined from an azimuthal diffuse reflectance calibration curve, K_{B} is Boltzmann constant (1.38×10⁻²³ J/K), T is absolute temperature in Kelvin, ω is an angular frequency, and α is a log-log slope of the calculated MSD curve.

20. The method of claim 16 further comprising one or more of:
outputting the measured one or more rheologic property;
calculating and outputting a determination based at least in part on the measured one or more rheologic property; and
calculating and outputting a recommendation based at least in part on the measured one or more rheologic property.
